(19) United States
(12) Patent Application Publication
(10) Pub. No.: US 2003/0078555 A1
(43) Pub. Date: Apr. 24, 2003

(54) Body-activated adhesive compositions, films, personal care articles and pantiliners

(76) Inventors: John Kerins, Neenah, WI (US); Dave A. Soerens, Neenah, WI (US); Richard W. Tanzer, Neenah, WI (US); William S. Pomplun, Seven Lakes, NC (US)

Correspondence Address: JOHN S. PRATT, KILPATRICK STOCKTON LLP (KIMBERLY CLARK), 1100 PEACHTREE STREET, SUITE 2800, ATLANTA, GA, 30309, US

(21) Appl. No.: 10/267,015

(22) Filed: Oct. 8, 2002

Related U.S. Application Data
(63) Continuation-in-part of application No. 09/222,417, filed on Dec. 29, 1998, now abandoned.

(51) Int. Cl. A61F 13/15; A61F 13/20

(52) U.S. Cl. 604/385.03; 604/372; 604/387

(57) ABSTRACT

Body-activated adhesives having adhesive properties which increase with exposure to temperature and humidity approximating the temperature and humidity proximate to the human body, particularly the temperature and humidity of the human crotch. The adhesive compositions are useful as adhesive coatings or layers in various personal care products, such as pantiliners. Pantiliners containing the adhesive compositions can be manufactured without a peel strip.

### CROSS REFERENCE TO RELATED APPLICATION

Priority is claimed to U.S. application Ser. No. 09/222,417, filed Dec. 29, 1998.

### FIELD OF THE INVENTION

The present invention relates generally to adhesive compositions. The adhesive compositions of the present invention are body activated in that the adhesive compositions of the present invention increase in peel adhesion strength with exposure to the warm and humid environment proximate the human body. Specifically, the adhesive compositions of the present invention increase in peel adhesion strength with exposure to temperatures and humidities greater than typical room storage conditions, about 70° F. (or about 20° C.) and 50 percent relative humidity. More specifically, the adhesive compositions increase in peel adhesion strength with exposure to temperatures and humidities greater than 80° F. (or about 26° C.) and 70 percent relative humidity. This invention also relates to moisture-sensitive adhesive compositions.

### BACKGROUND OF THE INVENTION

Residents of industrialized countries consume large quantities of materials. Much of the consumed materials is disposable and is discarded. The discarded materials are collected and sent to a landfill. As the number of industrialized countries increases and the population increases, the quantity of material consumed and eventually disposed of in landfills is expected to increase. Thus, it would be desirable to decrease the amount of material accumulating in landfills.

In the United States, the accumulation of municipal waste in landfills per person per year consists of approximately: 265 kilograms (hereinafter kg) paper and cardboard, 70 kg plastics, 65 kg metals, and 270 kg of various other materials. Contrary to popular belief, paper, although degradable, degrades slowly in a landfill environment. Headlines of newspapers can be legible after twenty-five years of burial in a landfill. Plastics, though making up a smaller percentage of accumulated waste material than paper, make up a more visible portion of the accumulated landfill material. Conventional plastics degrade more slowly than paper and are more visible and thus considered offensive. Therefore, it would be desirable to eliminate as much paper and plastic as possible from disposal in landfills.

Feminine protection pads, sanitary napkins and pantiliners are disposable, personal care products comprising paper and plastic components. These personal care products are used only once and then disposed. The amount of disposed personal care products will increase as the general population and awareness in personal hygiene increases. Currently, pantiliners 6 comprise a body-side liner 1, an absorbent pad 2, an outer cover sheet 3, a hot-melt, pressure-sensitive, garment attachment adhesive 4 and a release strip 5 as illustrated in FIGS. 1 through 5. A consumer removes the release strip 5 from the pantiliner to expose the underlying garment attachment adhesive. The underlying garment attachment adhesive is then used to adhere the pantiliner to the crotch of the consumer's undergarment. After the release strip 5 is removed, the release strip 5 is discarded in a trash receptacle and eventually disposed of in a landfill.

The conventional release strip 5 is a paper strip with a silicone-based release coating. The silicone-based coating is used to ensure that the release strip 5 easily peels away from the pantiliner without removing any of the underlying adhesive 4 from the pantiliner. Unfortunately, the silicone-based coating on the paper release strip 5 increases the water resistance of the paper release strip 5 and also decreases the degradability of the paper release strip 5. If flushed in a toilet, a conventional release strip 5 is not likely to break up in the toilet or the attached drain line, and is likely to cause clogging somewhere in the septic or sewage systems. If the release strip 5 does not clog the septic system or sewage transport system, but makes it to the sewage system, it will likely end up being collected on sewage treatment plant bar screens. Alternatively, the release strip may accumulate in the septic system causing plugging and flooding. It would be desirable, both economically and environmentally, to eliminate release strips, especially silicone-coated paper release strips. Furthermore, it would be desirable to provide a pantiliner not requiring a release strip. Therefore, there is a need to provide adhesives not requiring the use of release strips.

### SUMMARY OF THE INVENTION

The present invention provides adhesive compositions and films and articles incorporating the adhesive compositions. The adhesive compositions of the present invention are body activated in that the adhesive compositions increase in adhesive strength with exposure to the warm and humid environment proximate the human body. More particularly, the adhesive compositions of the present invention have the property of increasing in adhesive strength after exposure to temperatures greater than about 70° F. (or about 20° C.) and relative humidities greater than about 50 percent. Even more particularly, the adhesive compositions of the present invention increase in adhesive strength with exposure to temperatures greater than about 80° F. (or about 25° C.) and relative humidities greater than about 70 percent. Even more particularly, the adhesive composition of the present invention increase in adhesive strength with exposure to temperatures from greater than about 70° F. to about 110° F., and a relative humidity greater than about 50 percent to about 100 percent. Even more particularly, the adhesive compositions of the present invention increase in adhesive strength with exposure to temperatures from about 80° F. to about 1110° F., and a relative humidity greater than about 70 percent relative humidity. Even more particularly, the adhesive compositions of the present invention increase in adhesive strength with exposure to temperatures from about 90° F. to about 110° F., and a relative humidity greater than about 75 percent relative humidity. The adhesive compositions of the present invention increases in adhesive strength to fabrics, including, but not limited to, cotton and nylon, upon exposure to a warm and humid environment. In particular, the adhesive composition of the present invention increase in adhesive strength to cotton or nylon fabric with exposure to temperatures greater than 70° F. and relative humidities greater than 50 percent. Even more particularly, the adhesive composition of the present invention increase in adhesive strength to cotton or nylon fabric with exposure to a warm and humid environment ranging from about 90° F. to about 110° F., and greater than about 75 percent relative humidity.

Advantageously, the adhesive compositions of the present invention have little or no adhesion (herein defined as no peel adhesion strength to itself at standard room conditions) at typical room storage conditions (about 70° F. and 50 percent relative humidity). Thus, the adhesive compositions can be used to manufacture articles incorporating adhesive layers and coatings, without necessarily including protective coatings, strips or packaging that are normally included to protect the adhesive layers and coatings. The adhesive compositions of the present invention are especially useful as adhesive layers and coatings in personal care articles. In one illustrative application, the personal care article incorporating the body-activated adhesive compositions of the present invention is a pantiliner. The adhesives of the present invention allow the deletion of expensive, silicone-based peel paper strips and hot-melt, pressure-sensitive, garment adhesives used in conventional pantiliners. A pantiliner is described herein, which incorporates the adhesive compositions of the present invention, and does not require a silicone-based peel paper strip, eliminating, or at least minimizing, the amount of hot-melt, pressure-sensitive, garment adhesive required for its manufacture. The adhesives of the present invention may also be used to adhere other objects to a surface.

The films, coatings and articles, incorporating films and coatings of the adhesive compositions of the present invention, build adhesion to undergarment fabrics, particularly cotton, with exposure to warm and humid conditions approximating the environment proximate the human body, particularly the warm and humid environment of a human crotch. Because films and coatings of the adhesive compositions have low initial peel adhesion strength, articles incorporating these adhesive films and coatings do not require a peel strip. In addition, if there is a possibility of premature adhesion of adhesive layers during the manufacturing, packaging or storage of articles that incorporate the adhesive compositions of the present invention, an anti-blocking agent can be used to lightly coat the exposed surface of adhesive compositions. An anti-blocking agent is any material that provides a sufficient special separation of the adhesive layers, and thus prevents the adhesive layers from coming into close contact despite an increase in temperature and an increase in relative humidity.

The adhesive compositions of the invention include adhesives that have low initial adhesion and adhesive strength, but increase in adhesive strength with exposure to warm and humid environments. Such adhesives are referred to as body-activated adhesives. The adhesive compositions of the present invention also include moisture sensitive adhesives that increase in adhesive strength upon exposure to moisture (high humidity).

The response of the present adhesive compositions to warm and humid environments is attributed to increased molecular mobility and resultant local flow of the adhesive to make better, more intimate contact with the substrate. At high humidity, enough water vapor enters the adhesive to plasticize or hydrate a polymer component, and at higher temperature, the plasticized or hydrated polymer becomes more motile and is able to form a better adhesive bond with the substrate. This motility is associated primarily with the amorphous regions of the polymer, where the disordered polymer chains are more susceptible to localized rearrangement and repositioning (note this is a completely different mechanism than the first-order phase transition of crystalline melting cited in Stewart). Increased motility might typically be evidenced by a decrease in the glass transition temperature of a water-plasticized polymer compared to the same polymer completely dried. At the same time the adhesive retains enough cohesive strength to peel cleanly from the substrate.

Suitable adhesive compositions may comprise polymers and copolymers of ethylene oxide, including graft copolymers of poly(ethylene oxide); polyesters and copolyesters, including sulfonated polyesters and sulfonated copolyesters; polymers of vinyl acetate; copolymers of vinyl acetate and ethylene; and mixtures of the same. Graft copolymers of poly(ethylene oxide) include, but are not limited to, 2-hydroxyethyl methacrylate grafted copolymer of poly(ethylene oxide).

The adhesive compositions may also comprise moisture-sensitive polyesters, moisture-sensitive copolyesters, moisture-sensitive sulfonated polyesters, moisture-sensitive sulfonated copolyesters, and mixtures of the same. Moisture in the term “moisture-sensitive” refers to the moisture or water vapor present in the atmosphere.

The adhesive compositions may also include cohesive agents, tackifier resins and other known additives for adhesive compositions. Suitable cohesive agents include, but are not limited to, thermoplastic elastomeric cohesive agents comprising polymers or copolymers of vinyl acetate. Suitable tackifier resins include, but are not limited to, rosin ester tackifier resins. The adhesive compositions of the present invention may also comprise antioxidants, UV stabilizers, colorants, fillers, plasticizers, other adhesives that are not body-activated adhesives, and other known additives for adhesive compositions.

The adhesive compositions of the present invention may comprise from about 40 weight percent to about 90 weight percent of a polymer, copolymer or mixture thereof; from about 10 weight percent to about 60 weight percent of a cohesive agent; and from about 0 weight percent to about 50 weight percent of a tackifier resin. More specifically, the body-activated adhesive composition of the present invention may include from about 50 weight percent to about 89.5 weight percent of a polymer, copolymer or mixture thereof; from about 10 weight percent to about 30 weight percent of a cohesive agent; and from about 0.5 weight percent to about 20 weight percent of a tackifier resin.

The adhesive compositions of the present invention may be used to adhere objects to a surface. A method of adherence may include, but is not limited to, applying an adhesive composition of the present invention to an object, contacting the object to a surface, and exposing the adhesive composition to a temperature greater than 70° F. and a relative humidity greater than 50 percent. In particular, the body-activated adhesive compositions of the present invention increase in adhesive strength after 60 minutes of exposure to a temperature greater than 70° F. and a relative humidity greater than 50 percent. Even more particularly, the body-activated adhesive compositions of the present invention increase in adhesive strength after 15 minutes of exposure to a temperature greater than 70° F. and a relative humidity greater than 50 percent.

### BRIEF DESCRIPTION OF THE FIGURES

FIG. 1 is a partial cut-away top view of a prior art pantiliner.

FIG. 2 is a side view of a prior art pantiliner.

FIG. 3 is an end view of a prior art pantiliner.

FIG. 4 is a cross-section view of a prior art pantiliner taken through the section Y-Y in FIG. 1.

FIG. 5 is a bottom view of a prior art pantiliner.

FIG. 6 is a cross-section view of a pantiliner in accordance with one embodiment of the present invention described in Example 4.

FIG. 7 is an exploded view of the components of a pantiliner of FIG. 6 and Example 4, further including conventional garment attachment adhesive.

FIG. 8 is an enlarged, cross-section view of a pantiliner folded in a bi-fold relationship with the body-activated adhesive facing inward.

FIG. 9 is an enlarged, cross-section view of a pantiliner in accordance with one embodiment of the present invention described in Example 5; the pantiliner is folded in a bi-fold relationship with the anti-blocking agent facing inward.

### DETAILED DESCRIPTION OF THE INVENTION

The adhesive compositions of the present invention increase in adhesive strength when exposed to elevated temperature and humidity, particularly elevated temperature and humidity conditions approximating the temperature and humidity next to a mammal's skin and body. More particularly, the adhesives of the present invention increase in adhesive strength when exposed to the temperature and humidity proximate a human's skin.

The adhesive compositions of the present invention have particular utility in adhering articles where such conditions may be encountered.

These adhesive compositions may be useful for manufacturing, or adhering, various products, particularly personal care articles, and more particularly pantiliners. Other suggested articles include: sanitary napkins, feminine pads, diapers, incontinence devices, bandages, wound dressings, shoe inserts, etc.

Currently, conventional hot-melt adhesives are used to temporarily adhere pantiliners to the undergarment of a consumer. Some pantliners are held to undergarments by mechanical fasteners, such as VELCR®. Conventional adhesives are tacky at room temperature and require a peel strip to protect the adhesive surface prior to use. The adhesive is used to secure the pantiliner to the crotch of the consumer's undergarment during normal use. Desirably, the adhesive secures the pantiliner in a desired position during use, yet allows the pantiliner to be easily removed after use. Most desirably, the adhesive peels away with the pantiliner upon removal by the consumer, and transfers, little if any, adhesive to the undergarment to which the pantiliner was adhered. In order to perform in this manner, the adhesive should have better cohesion to the pantiliner than adhesion to the undergarment fabric. About, ninety-five percent of all women's undergarment crotch fabrics are a cotton fabric. Therefore, it would be desirable to develop adhesive compositions that adhere well to cotton fabric during human use conditions, but that are not tacky prior to use. Specifically, it would be desirable to develop adhesives that are not tacky under storage and room temperature and humidity conditions.

The present invention provides adhesives and pantiliners that meet many of the above requirements, by utilizing an adhesive composition that possesses beneficial properties. The adhesive compositions have low initial adhesive strength, and develop increasing adhesive strength during exposure to the temperature and humidity conditions encountered in normal pantiliner use. Specifically, the adhesive compositions of the present invention are sensitive to both temperature and moisture and activatable, exhibiting increasing adhesive strength upon exposure to the warm, humid environment next to human skin. More particularly, the adhesive compositions exhibit increasing adhesive strength upon exposure to warm, humid environments approximating the environment proximate a human body, about 100° F. and about 80 percent relative humidity.

Pantiliners are designed to absorb small amounts of liquids, usually one milliliter or less of menses and/or urine. To increase the comfort of the wearer, the pantiliner should not only absorb the liquids, but also wick the absorbed liquids away from the skin of the wearer. Desirably, a pantiliner also prevents the absorbed liquids, menses and/or urine, from soiling the undergarment to which the pantiliner is adhered. More desirably, the user should not be able to see the absorbed liquids through the reverse side of the pantiliner. Thus, pantiliners should have both absorbent and barrier properties.

The present invention provides a pantiliner incorporating a body activate adhesive composition of the present invention. One such pantiliner is illustrated in FIG. 6 and described in further detail in Example 4. The pantiliners take advantage of the beneficial properties of the adhesive compositions of the present invention, and also possesses the following advantages. The pantiliners comprise a body-activated adhesive that has excellent adhesion to undergarment crotch materials, particularly, cotton, which comprises about 95 percent of all undergarment crotch materials. Although the adhesive has excellent adhesion under moist body-temperature conditions, it is easily peeled away by the wearer after use, and leaves no visible adhesive on the undergarment after removal.

In at least one embodiment, the adhesive compositions are water soluble or water dispersible. The term “water-soluble” refers to an adhesive composition that is completely soluble in water. The term “water-dispersible” refers to an adhesive composition that breaks apart into smaller particles upon contact with water, and may eventually dissolve over time. Thus, the adhesives can be dissolved, and then removed by laundering with water. More importantly, any adhesive that may remain on a surface to which the adhesive was adhered may be cleaned or laundered with water. Desirably, the adhesive compositions are soluble or dispersible in tap water, but not in human wastes. In at least one embodiment, the adhesive compositions are resistant to urine and menses, yet are disintegratable or soluble in tap water.

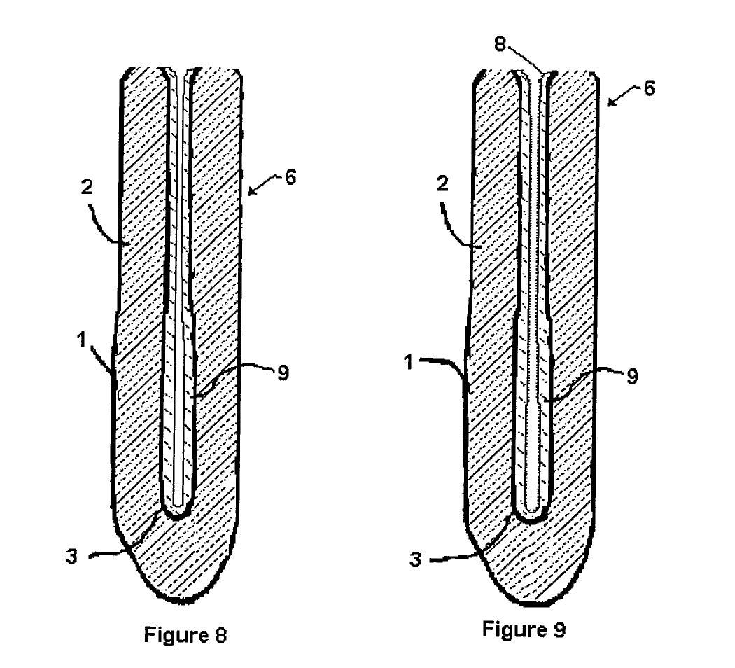

Advantageously, a pantiliner incorporating a body-activated adhesive of the present invention can be folded upon itself in a bifold relationship, as illustrated in FIG. 8. A folded pantiliner requires a smaller package. It may be desirable to provide at least some conventional pressure-sensitive adhesive 4, as illustrated in FIG. 7, to initially adhere the pantiliner, or any other article incorporating the body-activated adhesive, until the body-activated adhesive gains sufficient adhesive strength. It may be desirable to provide folded pantiliners with at least one spot of conventional pressure-sensitive adhesive 4 that contacts the body-activated adhesive on the opposite folded half, until unfolded by a consumer. The conventional pressure-sensitive adhesive may be protected by a small peel strip(s), particularly if the pantiliner is to be packaged in an unfolded relationship.

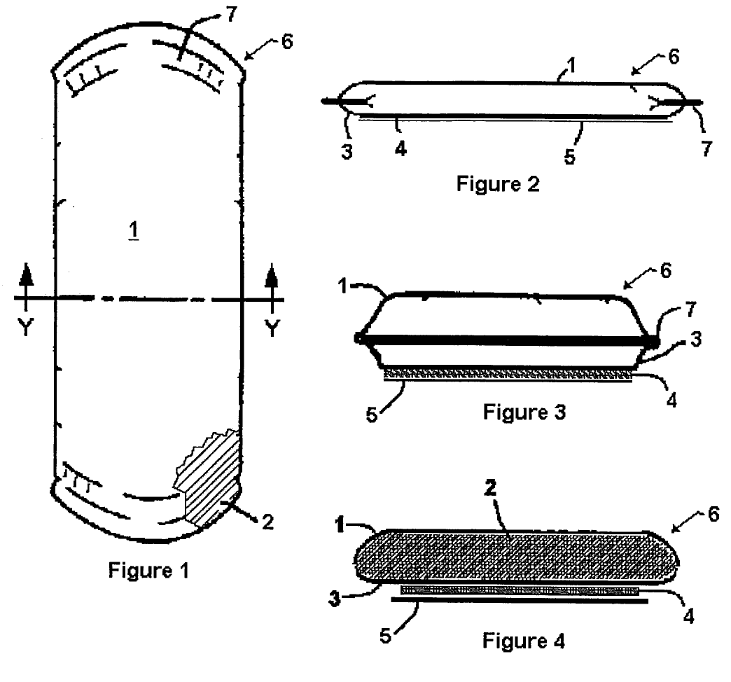

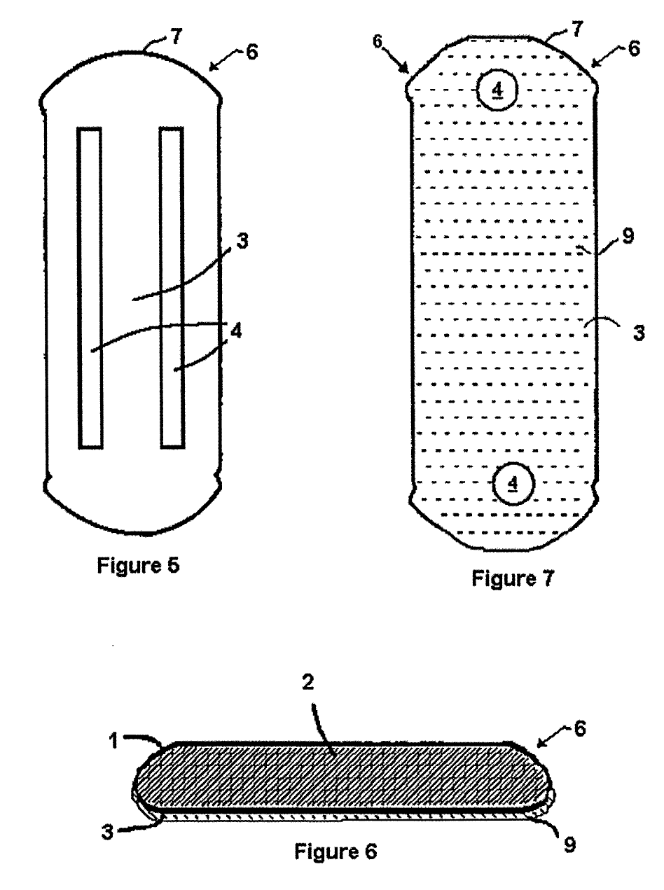

In some modes of the invention, the body-activated adhesive may prematurely become tacky. This may occur during manufacturing, shipping or storage when ambient temperature or humidity conditions approach, or even exceed, the activating temperature and humidity. Under such conditions it is possible for the adhesive surfaces of a pantiliner to stick to an adjacent surface (distal from the outer cover sheet). This may occur, for example, in a bi-fold configuration, or if stacked in a package. One method of mitigating this premature adhesion is to lightly coat the exposed surface of the body-activated adhesive with an anti-blocking agent, such as a powder, short fibers, or any material that will prevent the adhesive layers from coming into close contact. An anti-blocking agent is any material that provides a sufficient spatial separation of the adhesive layers, and thus prevents the adhesive layers from coming into close contact despite an increase in temperature and an increase in relative humidity. These agents are usually particulate in nature or short fibers. The pantiliners, incorporating a body-activated adhesive illustrated in FIGS. 6, 7, 8 and 9, are based on an absorbent core 2 and incorporate a body-activated adhesive 9. The body-activated adhesive does not necessarily have to be incorporated as a layer or coating, as illustrated in FIGS. 6, 7, 8 and 9, and can be applied in various manners and patterns. For example, the body-activated adhesive may be incorporated in a discontinuous pattern, such as the strips illustrated in FIG. 1, applied in a melt blown or in a discontinuous printed pattern. The pantiliners may include additional layers, for example, a support layer (not illustrated), intermediate the adhesive layer 9 and the absorbent core 2. The pantiliner may further comprise additional absorbent layers, a body-side liner, etc.

The adhesive compositions of the invention include adhesives that have low initial adhesion and adhesive strength, but increase in adhesive strength with exposure to warm, humid environments. Such adhesives are referred to herein as body-activated adhesives. These body-activated adhesives include polymers and copolymers of ethylene oxide, desirably graft copolymers of poly(ethylene oxide). Desirable graft copolymers of poly(ethylene oxide) and methods of making these graft copolymers of poly(ethylene oxide) are disclosed in U.S. Pat. Nos. 6,117,947 and 6,171,117 and U.S. application Ser. No. 09/001,408, the disclosures of which are incorporated herein by reference in their entirety. Based on the results in Examples 1, 2 and 3 herein, it is anticipated that grafted and ungrafted poly(ethylene oxide) compositions (hereinafter PEO) will provide the desired body-activated adhesive properties. For example, ungrafted PEO resins, such as POLYOX® WSR N-750 and POLYOX® WSR N-80, may be used to replace the grafted PEO in Examples 1, 2 and 3. Similarly, other grafting monomers could be substituted as suggested in the above-referenced patent applications. These graft copolymers are generally branched, that is they have pendent side groups extending from a “back bone” structure. They may further be characterized by having the majority of the mass of the polymer in the “back bone” structure. Desirably less than 40% of the polymer mass consists of side chains. Alternatively less than 30% of the polymer mass consists of side chains. Furthermore, while there may be some cross-linked fraction in the polymers, it should be only to the extent that any gel fraction of the polymer does not interfere with extrusion or application of the adhesive formulation.

Additional body-activated adhesive compositions may include the polyesters described in U.S. Pat. Nos. 5,543,488, 5,552,495 5,709,940 and 5,718,790, the disclosures of which are incorporated herein by reference in their entirety. Commercial examples of these polyesters can be obtained from Eastman Chemical Company of Kingsport, Tenn. and include Eastman resins AQ 1350 and AQ 1950. Other examples of body-activated adhesive polyesters include the water-sensitive sulfonated polyesters described in European Patent Application EP 0 761 795 A2.

These sulfonated polyesters contain 2 to 25 mole percent of one or more sulfomonomers containing at least one metallic sulfonated group, or a nitrogen containing non-metallic sulfonated group attached to an aromatic or cycloaliphatic nucleus, and one or more functional groups selected from hydroxyl, carboxyl or amino. These sulfonated polyesters also comprise one or more monomers containing a difunctional carboxylic acid or the corresponding methyl ester; one or more difunctional reactants comprising a glycol or a mixture of a glycol and a diamine; and optionally, one or more difunctional reactants selected from hydroxycarboxylic acids, aminocarboxylic acids, amino-alcohols, or mixtures thereof; and optionally, a multifunctional reactant containing at least three functional groups selected from hydroxyl, carboxyl, or mixtures thereof. One commercial example of a sulfonated polyester with body activated adhesive properties was obtained from National Starch and Chemical Company of Wilmington, Del., and provided as National Starch resin 70-4395.

The body-activated adhesive compositions of the present invention may also comprise various additives. Desirably, the PEO containing body-activated adhesive compositions of the present invention comprise cohesive agents and tackifier resins. The adhesive compositions of the present invention may also comprise antioxidants, UV stabilizers, colorants, fillers, plasticizers and other known additives for adhesive compositions. Desirably, the PEO based adhesive compositions of the present invention comprise about 10 to about 60 weight percent elastomeric cohesive agent, more desirable about 10 to about 30 weight percent. The cohesive agent should be elastomeric to give additional cohesive strength. Additionally, it is desirable that the elastomeric cohesive agent is thermoplastic so that it can be easily melt blended into the adhesive compositions. In general, the melt flow rate of the cohesive agent should be greater than 0.2 grams per 10 minutes and less than 20 grams per 10 minutes based on ASTM standard D1238. Suitable cohesive agents include, but are not limited to, ethylene/vinyl acetate copolymers such as the LEVAPRIN® 600 ethylene/vinyl acetate copolymer employed in Examples 1, 2 and 3. LEVAPRIN® 600 ethylene/vinyl acetate copolymer has a melt flow rate of less than about 5 grams per 10 minutes at 190° C. Desirable ethylene/vinyl acetate copolymers include ethylene/vinyl acetate copolymers with a vinyl acetate contents ranging from about 20 to 100 percent vinyl acetate, and include VINAC® B-100 vinyl acetate homopolymer, acid-modified EVA resins such as BYNEL® resins and ethylene/acrylic or methacrylic acid copolymer resins such as NUCREL® from DuPont. In addition, other thermoplastic elastomers, such as KRATON 1107 available form Shell Chemical Company and the like, may be suitable cohesive agents.

Accordingly, the body-activated adhesive composition of the present invention may include from about 40 weight percent to about 90 weight percent a graft copolymer of ethylene oxide, from about 10 weight percent to about 60 weight percent of a cohesive agent, and from about 0 weight percent to about 50 weight percent of a tackifier resin. More specifically, the body-activated adhesive composition of the present invention may include from about 50 weight percent to about 89.5 weight percent a graft copolymer of ethylene oxide; from about 10 weight percent to about 30 weight percent of a thermoplastic, elastomeric cohesive agent comprising a polymer or copolymer of vinyl acetate; and from about 0.5 weight percent to about 20 weight percent of a rosin ester tackifier resin

Additionally, a wide variety of tackifier resins may be incorporated into the body-activated adhesive compositions of the present invention. Suitable tackifier resins include, but are not limited to, FORAL® 85, a slightly polar rosin ester tackifier resin employed in Example 1, and PICCOTEX® 75, a vinyl toluene-co-alpha methyl styrene copolymer employed in Example 2. Any tackifier resin that provides the proper level of adhesion under body-wear conditions, but does not generate appreciable tackiness under normal storage, is suitable. Other suggested tackifier resins include other rosin esters, aromatic esters and terpene phenol resins.

The body-activated adhesive films described herein have excellent adhesion to cotton undergarment crotch materials under moist body-temperature conditions. Adhesive films and coatings comprising the compositions described herein eliminate the need for peel paper strips, and also eliminate or minimize the need for conventional garment attachment adhesives. This unexpected advantage of the body-activated adhesive compositions described herein allows a reduction in the cost and the number of materials required to manufacture personal care articles, including sanitary napkins and pantiliners.

Other features and advantages of the present invention will become apparent after a review of the above Detailed Description and the following disclosure of four exemplary embodiments of body-activated adhesive compositions in Examples 1 through 5 below.

### EXAMPLE 1

### Body-Activated Adhesive Composition

The body-activated adhesive composition of Example 1 was prepared from a melt blend of the following three components in the following proportions: 70 weight percent of a poly(ethylene oxide) (hereinafter PEO) graft copolymer; 10 weight percent of ethylene/vinyl acetate copolymer; and 20 weight percent of rosin ester tackifier resin. The PEO graft copolymer that was used in this and the following examples was produced by the reactive extrusion of: 14.8 pounds of POLYOX® 205 PEO resin; 1.5 weight percent of 2-hydroxyethyl methacrylate (hereinafter HEMA) monomer, relative to the weight of the PEO resin; 0.15 weight percent LUPERSOL® 101 free radical peroxide initiator; and 0.2 pounds of titanium dioxide pigment. The base PEO resin used to produce the PEO graft copolymer was POLYOX® 205, a commercially available polymer of ethylene oxide with a reported average molecular weight of 600,000 grams per mole. Various molecular weights of PEO resins can be obtained from Union Carbide Corporation, and are sold under the trademark POLYOX®. LUPERSOL® 101 is a free radical initiator comprising 2,5-dimethyl-2,5-di(t-butylperoxy) hexane, and is commercially available from Elf Atochem North America, Inc. of Philadelphia, Pa. The free radical initiator is used to initiate sites on the PEO for grafting the HEMA monomer onto the PEO. PEO graft copolymers and methods of making these PEO graft copolymers are disclosed in U.S. Pat. Nos. 6,117,947 and 6,171,117 and U.S. application Ser. No. 09/001,408. Compositions comprising these PEO graft copolymers are useful as body-activated adhesives. The HEMA-grafted-PEO also contained the following stabilizing agents: 1000 parts per million (hereinafter ppm) of IRGANOX® 1010 and IRGANOX® 1076, and 2000 ppm IRGANOX® 168.

The ethylene/vinyl acetate copolymer of the blend was a copolymer of 60 percent vinyl acetate and 40 percent ethylene that is commercially available from Bayer Corporation under the trademark LEVAPRIN® 600. The rosin ester tackifier resin component of the blend was obtained from Hercules Corporation and is sold under the trademark FORAL® 85. The above components were melt blended in a HAAKE TW-100 twin-screw extruder with an intensive mixing screw at a temperature of 180° C. through a strand die in order to produce the body-activated adhesive formulation of Example 1. The extruded formulation was cooled and then pelletized. The resulting pellets were remelted in the HAAKE TW-100 twin-screw extruder at 180° C. and extruded through a film die onto a 2 mil MYLAR polyester film. The coating thickness of the body-activated adhesive on the 2 mil MYLAR® polyester film was also 2 mils. The resulting “tape” formed from the MYLAR® polyester film and body-activated adhesive coating was used to test the adhesive properties of the body-activated adhesive composition, the results of which are reported in Table 1 below. All of the tapes produced in this example and the following examples had low peel adhesion strength at room temperature, as indicated by the ability to unwind the rolls without the use of a release liner.

### EXAMPLE 2

### Body-Activated Adhesive Composition

The body-activated adhesive composition and tapes of Example 2 were prepared in the same manner as described in Example 1 above, except that the body-activated adhesive composition of Example 2 was prepared from a melt blend of the following three components in the following proportions: 76.7 weight percent of the aforementioned HEMA-grafted-PEO; 16.7 weight percent of LEVAPRIN® 600 ethylene/vinyl acetate copolymer; and 6.6 weight percent of PICCOTEX® 75, a vinyl toluene-co-alpha methyl styrene copolymer, available from Hercules Corporation.

### EXAMPLE 3

### Body-Activated Adhesive Composition

The body-activated adhesive composition and tapes of Example 3 were prepared in the same manner as described in Example 1 above, except that the body-activated adhesive composition of Example 3 was prepared from a melt blend of only two components, the PEO component and the cohesive agent component. The two components of Example 3 were melt blended in the following proportions, 90 weight percent of aforementioned HEMA-grafted-PEO and 10 weight percent of LEVAPRIN® 600 ethylene/vinyl acetate copolymer. Example 3, in addition to illustrating another exemplary body-activated adhesive formulation, also demonstrates the optionality of the tackifier component in the body-activated adhesive compositions of the present invention.

Peel Adhesion of Examples 1-3 from Cotton Cloth After Exposure to Conditions Simulating the Human Crotch Environment

To simulate the adhesion of a pantiliner incorporating an adhesive layer of the body-activated adhesive compositions to the cotton crotch of an undergarment, tapes of each of three examples were cut to a one-inch width and applied to standardized cotton fabric. Each tape/fabric sample was then placed on a metal plate perforated with 7-millimeter diameter holes spaced 10 millimeters apart, center to center, and exposed to humid air approximating human crotch conditions. A brass block 2.4375 inches wide by 4.75 inches long by 1 inch high, and weighing 1600 grams was placed on top of the tape/fabric sample to supply a pressure load of approximately 0.3 pounds per square inch (psi) to the tape/fabric sample.

Five tape/fabric samples of each example were placed under the 0.3 psi load and conditioned at 100° F. and 80 percent relative humidity for 15, 60 and 180 minutes. The tape/fabric samples were tested immediately after each exposure period on a Sintech tensile tester in the “T-peel” mode at a crosshead speed of 20 inches per minute. The average peel adhesion was recorded for each of the five tape/fabric samples at each of the three exposures, for each of the examples. The average peel adhesion for each of the exposure periods and examples was calculated and is reported in Table 1 below.

TABLE 1
PEEL ADHESION TO COTTON FABRIC

| | Example | Example | Example |
|---|---|---|---|
| Conditioning | 1 | 2 | 3 |
| Time (min) | (grams/inch) | (grams/inch) | (grams/inch) |
| 15 | 63 | 14 | 11 |
| 60 | 73 | 89 | 17 |
| 180 | 156 | 100 | 49 |

The data presented in Table 1 demonstrate a distinguishing feature of the adhesive compositions of the present invention, and films, coatings, articles, etc. comprising the adhesive compositions of the present invention. The adhesive compositions and films, coatings, articles, etc., incorporating the adhesive compositions described herein, are body activated. That is, the adhesive compositions increase in adhesive strength with increasing exposure to the temperature and humidity conditions approximating the conditions next to human skin. Specifically, the adhesion of the adhesive films and tapes to cotton fabric builds at body temperature, about 100° F., and humidity, about 80 percent relative humidity.

Self Peel Adhesion of Examples 1 and 2 without Body Humidity Exposure

To demonstrate the low initial tack, or low adhesive strength, and the suitability of the body-activated adhesive compositions to be employed without release strips, the compositions of Examples 1 and 2 were tested under conditions approximate to normal room conditions. The tests were conducted as follows. A tape was constructed as described in Examples 1 and 2 above. Each of the tapes was then folded in half on itself with the adhesive surface of one folded half contacting the adhesive surface of the opposite folded half. The brass block used in the previous test procedure was placed on top of the folded tape to simulate possible compressive forces encountered in packaging. After three days of exposure at room temperature and humidity, of approximately 70° F. and 50 percent relative humidity, the brass block was removed. After the three days exposure under 0.3 psi load at room conditions, the tapes of Examples 1 and 2 unfolded readily, and no adhesive attachment was found. These results indicate the potential of utilizing the body-activated adhesives of the present invention for manufacturing and packaging pantiliners and other articles without release strips and additional single use packaging. Thus, articles substituting the adhesive compositions of the present invention for conventional adhesives can be manufactured without release strips at reduced material, manufacturing and environmental costs.

### EXAMPLE 4

### Pantiliner Comprising a Layer of Body-Activated Adhesive Having Optional Barrier Properties

To produce the pantiliners of Example 4, laminates were assembled from four components in the sequence illustrated in FIG. 6, and described in detail below. The pantiliners were assembled by laminating a body-side liner 1 to an absorbent core 2. The absorbent core 2 was a coform absorbent core formed from a 120 grams/square meter basis weight of a mixture of 60 percent by weight of wood fluff pulp and 40 percent by weight of a meltblown polymer. The meltblown polymer used in Example 4 was a polyamide obtained from H. B. Fuller Company under the trade designation NP-2068. The body-side liner 1 consisted of a bonded, carded web containing bicomponent polylactide fibers. Bicomponent polylactide fibers and methods of making the bicomponent polylactide fibers are disclosed in U.S. Pat. No. 5,698,322, the disclosure of which is incorporated herein by reference in its entirety.

A first laminate (laminate A) was prepared consisting of a body-side liner 1 and an absorbent core 2. A second laminate (laminate B) was prepared by coating an outer cover film 3 with body-activated adhesive 9. The two laminates were combined by bonding the absorbent core side of laminate A to the outer cover film side of laminate B. The outer cover film was constructed from a 30 μm thick monolayer film consisting of a water-dispersible blend of a PEO, a poly[ethylene-co-(acrylic acid)], and a plasticizer, in the weight ratios of 80/15/5, respectively. The PEO of Example 4 was obtained from Union Carbide, and is sold under the trade designation POLYOX® WSR N-80. The poly[ethylene-co-(acrylic acid)] was obtained from Dow Chemical Company, and is sold under the trade designation PRIMACOR 1410. The plasticizer of the Example 4 is a fatty acid amide plasticizer, and is sold under the trade designation TWEEN® 20. The body-activated adhesive layer 9 employed in Example 4 was formed by coating a layer ranging in thickness from about 10 μm to 50 μm of a sulfonated copolyester blend onto the outer cover film. The copolyester blend of Example 4 was provided by National Starch and Chemical Company under the trade designation 70-4395.

Although, the adhesive compositions of this and the preceding examples were applied as a continuous layer, it is understood that the body-activated adhesives of the present invention may be applied in other manners and patterns. For example, the adhesives may be applied in other known manners and patterns, including any discontinuous pattern such as stripes of adhesive as illustrated in FIG. 1, a swirl spray pattern, a meltblown process, a discontinuous printed pattern, etc. It is also understood that the pantiliner may contain a second adhesive that is not a body-activated adhesive, and that the amount of this second adhesive may be less than the amount of the body-activated adhesive.

The peel adhesion strengths of the adhesive layer and accompanying pantiliner of Example 4 to both cotton and nylon fabrics were tested after varying times of exposure in a controlled force-draft oven set at 37° C. and 80 percent relative humidity. The results of the peel adhesion strength tests of the pantiliners incorporating the body activated adhesive composition of Example 4 are presented in Table 2 below.

TABLE 2
PEEL ADHESION STRENGTHS OF PANTILINERS COMPRISING
THE BODY-ACTIVATED ADHESIVE COMPOSITIONS OF
EXAMPLE 4 TO COTTON AND NYLON UNDERGARMENT
FABRICS

| | Peel Adhesion | Peel Adhesion |
|---|---|---|
| Exposure | to Cotton | to Nylon |
| Time | Fabric | Fabric |
| (minutes) | (grams per inch) | (grams per inch) |
| 15 | 8 | — |
| 60 | 111 | — |
| 120 | 193 | — |
| 360 | 194 | 6 |

A pantiliner in accordance with the above detailed Example 4 was worn by a female subject for a period of eight hours. Some delamination of the pad was noted on removal. Thus, good adhesion to cotton undergarment fabric was achieved but exceeded the pad cohesion strength. However, delamination of the pantiliner may be prevented by either (1) increasing the cohesion strength of the pad to the adhesive, barrier film or (2) decreasing the adhesion strength of the layer to the undergarment fabric. By increasing the lamination adhesion above the adhesion of the body-activated adhesive to the undergarment, complete removal of the adhesive and pantiliner from the undergarment after use is ensured.

### EXAMPLE 5

### Pantiliner Comprising Body-Activated Adhesive and Anti-Blocking Agent

In some modes of this invention, the body-activated adhesive may prematurely become tacky. This may occur during manufacturing, shipping, or storage, when ambient temperature and humidity conditions approach or even exceed the activation conditions. Under such conditions, the pantiliners may stick to each other or to the packaging material. This is an undesirable condition.

One method of mitigating this premature tackiness is to lightly coat the exposed surface of the body-activated adhesive with an anti-blocking agent such as a powder or short fibers. These anti-blocking agents serve to temporarily defeat the adhesion of the underlying adhesive, by providing a small gap between the adhesive and the adjacent surface. FIG. 9 illustrates a pantiliner in a folded configuration illustrates a pantiliner 6 wherein the body-activated adhesive 9 is coated with an anti-blocking agent 8.

Various fibers, such as cotton linters and short cut (less than about 3 mm) polyester staple fibers may be flocked onto the exposed surface of the body-activated adhesive. Desirably, the fibers are blown onto the surface of the body-activated adhesive. Desirably, the fibers are blown onto the surface of the body-activated adhesive shortly after it is applied to the support layer. Only a light application of fibers, less than 10 grams per square meter, or alternatively, less than 5 grams per square meter is required for this purpose.

Alternatively, powders such as corn starch, talc, diatomaceous earth, microcrystalline cellulose, silica or alumina may be used as the anti-blocking agent. Powders which are capable of absorbing moisture while maintaining their flow properties and lubricity are particularly desirable. Silica gel is one example of such a powder.

When used by a consumer, the pantiliner is placed into an undergarment (with the adhesive side of the pantiliner in contact with the undergarment) and then the undergarment/pantiliner combination is donned. As the pantiliner is worn it is subject to a combination of temperature, humidity, pressure and movement. Under these conditions, the body-activated adhesive is sufficiently soft that the adhesive flows around the anti-blocking agents and a fresh surface of the adhesive, not contaminated by the anti-blocking agent, provides adhesion to the under garment.

The present invention has been illustrated in great detail by the above specific Examples. It is to be understood that these Examples are illustrative embodiments, and that this invention is not to be limited by any of the Examples or the Detailed Description. Those skilled in the art will recognize that the present invention is capable of many modifications and variations without departing from the scope of the invention. Accordingly, the Detailed Description and Examples are meant to be illustrative, and are not meant to limit in any manner the scope of the invention as set for the in the appended claims.

We claim:

1. A body-activated adhesive composition, wherein the composition comprises a graft copolymer of poly(ethylene oxide) or a sulfonated copolyester, and wherein the body-activated adhesive composition has little or no adhesion at room storage conditions, but increases in adhesive strength with exposure to a temperature greater than 70° F. and a relative humidity greater than 50 percent.

2. The body-activated adhesive composition of claim 1, wherein the adhesive composition increases in adhesive strength with exposure to a temperature greater than about 80° F. and a relative humidity greater than about 70 percent.

3. The body-activated adhesive composition of claim 1, wherein the adhesive composition increases in adhesive strength with exposure to a temperature greater than 70° F. and less than about 110° F., and a relative humidity greater than 50 percent and less than about 100 percent.

4. The body-activated adhesive composition of claim 1, further comprising a polyester.

5. The body-activated adhesive composition of claim 1, further comprising poly(ethylene oxide).

6. The body-activated adhesive composition of claim 1, further comprising a polymer of vinyl acetate.

7. The body-activated adhesive composition of claim 6, further comprising a copolymer of vinyl acetate and ethylene.

8. The body-activated adhesive composition of claim 1, wherein the adhesive composition further comprises:
a) from about 40 weight percent to about 90 weight percent of the graft copolymer of poly(ethylene oxide),
b) from about 10 weight percent to about 60 weight percent of a cohesive agent, and
c) from about 0 weight percent to about 50 weight percent of a tackifier resin.

9. The body-activated adhesive composition of claim 1, wherein the adhesive composition further comprises:
a) from about 50 weight percent to about 89.5 weight percent of the graft copolymer of poly(ethylene oxide);
b) from about 10 weight percent to about 30 weight percent of a thermoplastic, elastomeric cohesive agent comprising a polymer or copolymer of vinyl acetate; and
c) from about 0.5 weight percent to about 20 weight percent of a rosin ester tackifier resin.

10. The body-activated adhesive composition of claim 9, wherein the graft copolymer of poly(ethylene oxide) is a 2-hydroxyethyl methacrylate grafted copolymer of poly(ethylene oxide).

11. The body-activated adhesive composition of claim 1, wherein the adhesive composition further comprises a moisture-sensitive polyester, a moisture-sensitive copolyester, a moisture-sensitive sulfonated polyester, or a moisture-sensitive sulfonated copolyester.

12. An article comprising the body-activated adhesive composition of claim 1, wherein at least part of the exterior surface of the article is coated with the body-activated composition of claim 1.

13. The article of claim 12, wherein the body-activated adhesive composition is coated with an anti-blocking agent at a surface of the adhesive that is distal from the surface of the body-activated adhesive that is in contact with the exterior surface of the article.

14. A pantiliner comprising the body-activated adhesive of claim 1 and a second adhesive that is not a body-activated adhesive.

15. The pantiliner of claim 13, wherein the amount of the second adhesive that is less than the amount of the body-activated adhesive.

16. The body-activated adhesive of claim 1, wherein the adhesive has increased adhesive strength after 15 minutes of exposure to a temperature greater than 70° F. and a relative humidity greater than 50 percent.

17. The body-activated adhesive of claim 1, wherein the adhesive has increased adhesive strength after 60 minutes of exposure to a temperature greater than 70° F. and a relative humidity greater than 50 percent.

18. A method of adhering an object using the body activated composition of claim 6.

19. An adhesive composition comprising a polymer selected from a graft copolymer of poly(ethylene oxide) or a sulfonated copolyester, wherein the composition increases in adhesive strength to cotton or nylon fabric with exposure to a warm and humid environment proximate human skin.

20. The adhesive composition of claim 18, wherein the adhesive composition increases in adhesive strength to cotton or nylon fabric with exposure to a warm and humid environment ranging from about 90° F. to about 110° F., and greater than about 75 percent relative humidity.

21. A method of adhering an object to a surface comprising:
a) applying an adhesive composition comprising a graft copolymer of poly(ethylene oxide) or a sulfonated copolyester to an object,
b) contacting the object to a surface, and
c) exposing the graft copolymer of poly(ethylene oxide) or the sulfonated copolyester to a temperature greater than 70° F. and a relative humidity greater than 50 percent.
